(19) United States
(12) Plant Patent Application Publication
(10) Pub. No.: US 2004/0078857 P1
(43) Pub. Date: Apr. 22, 2004

(54) Skimmia japonica named 'Ranso En'

(76) Inventor: Noriaki Kogawa, Minia Tsukuda (JP)

Correspondence Address: CHRISTIE, PARKER & HALE, LLP, P.O. BOX 7068, PASADENA, CA, 91109-7068, US

(21) Appl. No.: 10/278,606

(22) Filed: Oct. 22, 2002

(51) Int. Cl. A01H 5/00

(52) U.S. Cl. PLT/226

(57) ABSTRACT

A new plant variety of Skimmia japonica characterized Cream variegated margins.

### BACKGROUND OF THE INVENTION

The present invention relates to a new and distinct variety of Skimmia japonica, which was found as a branch sport on a cultivated specimen of Skimmia japonica (unpatented). The varietal denomination of the new variety is ‘RANSO EN’.

The genus Skimmia is included in the family Rutaceae which comprises about 161 genera and 1700 species of evergreen and deciduous trees and shrubs, Asian in origin. Skimmia comprises approximately 6 species, originating in Western Himalayas, Eastern China and Japan, many of which possess desirable ornamental characteristics. Skimmia japonica is a native of Japan and Korea.

### SUMMARY OF THE INVENTION

The new variety was discovered as a branch sport and differs from its parent by having Cream colored variegated margins. Asexual reproduction of the new variety by cuttings, performed in Vacaville, Calif., have confirmed that the distinctive characteristics of the new variety are stable and transmitted to succeeding generations, and the new variety reproduces true to type.

### COMPARISON WITH PARENT

‘RANSO EN’ is distinguished from its parent and all other varieties of Skimmia japonica of which I am aware by having Cream variegated margins.

### BRIEF DESCRIPTION OF ILLUSTRATION

The accompanying illustration shows a specimen of the new cultivar in a photo illustration as true to color as is reasonably possible to make in an illustration of this character.

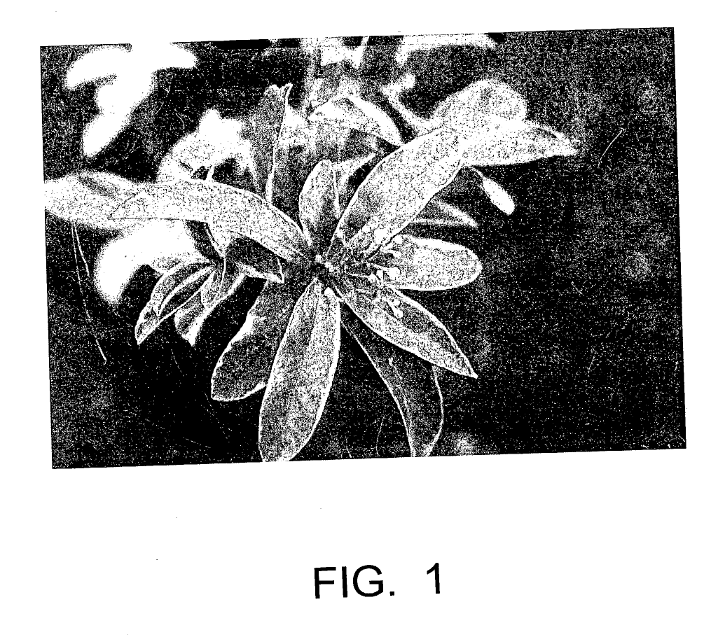

FIG. 1 illustrates the distinctive Cream colored marginal variegation.

### DESCRIPTION OF THE NEW VARIETY

‘RANSO EN’ has not been observed under all possible environmental, cultural and light conditions. The following observations and descriptions are of plants grown in Lewisberry, Pa. and Vacaville, Calif. In this description, color references are to The Royal Horticultural Society Colour Chart (2001) and terminology used in the color descriptions herein refers to plate numbers in this color chart. Phenotypic expression may vary with light intensity, cultural and environmental conditions.

Botanical classification: Skimmia japonica ‘RANSO EN’

Parentage.—Branch sport of Skimmia japonica (unpatented).

Propagation.—Vegetative cuttings.

Plant:

Size.—Between 50 to 60 cm tall.

Habit.—Upright to obliquely ascending, to spreading for older plants.

Branching:

Lateral Branches.—Upright to obliquely ascending, spreading on older plants. Typical Branch Quantity: About 200.

Observed branch.—Quantity: 10. Size: Between 50 to 60 cm long; Diameter about 6 mm.

Color.—Basal Branches: Near Greyed Group 194C. Apical Branches: Near Green Group 143C.

Color of new shoots.—Near Green Group 143C.

Leaf:

Shape.—Lanceolate to oblanceolate.

Size.—Length about 6.5 cm; width about 2.5 cm.

Arrangement.—Alternate, simple.

Margin.—Smooth, entire.

Texture/Substance.—Coriaceus, glabrous with a waxy surface covered with punctate glands.

Color: The following color readings are of specimens grown in Vacaville, Calif., 3 year old plants grown in 2 quart containers.

New foliage.—Upper side Center: Near Greyed-Green Group 191B. Upper side Margin: Near White Group 155B. Under side Center: Near Greyed-Green Group 191B. Under side Margin: Near White Group 155B.

Mature foliage.—Upper side Center: Near Greyed-Green Group 191B. Upper side Margin: Near White Group 155B. Under side Center: Near Greyed-Green Group 191B. Under side Margin: Near White Group 155B.

Venation pattern.—Upper Surface: Alternately pinnate, center vein depressed above. Under Surface: Side veins obscure, center vein raised beneath.

Petioles.—Size: Between 3 to 4 mm. Color: Near Greyed-Green Group 191B. Texture: Smooth.

Internodes.—Between 2 to 3 cm.

Hardiness.—USDA Zone 6 (−10° F. to 0° F.).

Vigor.—Slow to establish; moderate growth rate once established.

Pests/Diseases.—None Noted.

Infloresence:

Bloom period.—Outdoors: September to November. Greenhouse: Not observed in a greenhouse environment.

Form.—Cyme.

Sex of flowers.—All pistilate.

Size.—Between 4 to 8 cm.

Shape.—Ovoid.

Fragrance.—None noted.

Lastingness of individual blooms.—Between 2 to 3 weeks.

Sepals:

Quantity.—About 4.

Shape.—Broadly triangular.

Size.—Between 1 to 2 mm.

Fruit.—Mature fruit not yet observed.

I claim:

1. A new plant variety of Skimmia japonica of the variety substantially as shown and described.
